(19) United States
(12) Patent Application Publication
(10) Pub. No.: US 2003/0013097 A1
(43) Pub. Date: Jan. 16, 2003

(54) Genes overexpressed in prostate disorders as diagnostic and therapeutic targets

(76) Inventors: John Barnard Welsh, San Diego, CA (US); Garret Malcolm Hampton, San Diego, CA (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 10/054,498

(22) Filed: Jan. 22, 2002

Related U.S. Application Data
(60) Provisional application No. 60/301,639, filed on Jun. 28, 2001.
(60) Provisional application No. 60/263,461, filed on Jan. 23, 2001.

(51) Int. Cl. C12Q 1/68; G01N 33/574

(52) U.S. Cl. 435/6; 435/7.23

(57) ABSTRACT

Disclosed are methods for diagnosing, monitoring the progression of, and treating a prostate disorder based upon genes that are differentially expressed in prostate disorders. Also disclosed are methods for identifying agents useful in the treatment of a prostate disorder, methods for monitoring the efficacy of a treatment for a prostate disorder, methods for inhibiting the proliferation of a prostate cell, and prostate-specific vectors including the promoter of these genes.

### CROSS-REFERENCE TO RELATED APPLICATIONS

This application claims the benefit of U.S. provisional application No. 60/263,461 filed Jan. 23, 2001 and U.S. provisional application No. 60/301,639 filed Jun. 28, 2001. The aforementioned applications are incorporated herein by reference.

### BACKGROUND OF THE INVENTION

1. Field of the Invention

The present invention relates generally to genes useful as diagnostic markers and/or targets for therapeutic intervention in prostate disorders such as prostate cancer. More particularly, the present invention concerns the identification of differentially expressed genes in malignant and normal prostate tissues and methods of diagnosis, prognosis and treatment of prostate disorders based upon these genes.

2. Description of the Related Art

Prostate cancer is the most common malignancy in men and the second most frequent cause of cancer death in the United States. Its development proceeds through a series of defined states, including pre-invasive disease (prostatic intraepithelial neoplasia, PIN), invasive cancer and androgen-dependent or androgen-independent metastases as described, e.g., in Scher et al., Urology, Vol. 55, pp. 323-327 (2000). The early stages of organ-confined prostate cancer are generally curable by surgery or radiation therapy, and detection efforts based on prostate-specific antigen (PSA) screening as described, e.g., in Catalona et al., JAMA, Vol. 270, pp. 948-954 (1993), have led to the identification of thousands of men with localized disease. Although serum PSA is widely recognized as the best prostate tumor marker currently available as described, e.g., in Brawer, Semin. Surg. Oncol., Vol. 18, pp. 3-9 (2000), screening programs utilizing PSA alone or in combination with digital rectal examination have failed to improve the survival rate of men with prostate cancer.

Several disadvantages attend the use of PSA as a diagnostic marker. First, while PSA is specific for prostate tissue, it is produced by normal as well as malignant prostate tissue, and quantification of PSA expression in a fragment of prostate tissue does not unambiguously classify that tissue with respect to malignancy or malignant potential. Second, not every prostate tumor secretes PSA. Third, while high PSA serum levels are an effective indicator of prostate cancer, modestly elevated levels, e.g., between 4 and 10 ng/mL are seen in men with obstructive or inflammatory uropathies, lowering the specificity of PSA as a cancer marker as described, e.g., in Brawer et al., Am. J. Clin. Pathol., Vol. 92, pp.760-764 (1989). Other biomarkers such as glandular kallikrein 2 (hK2) and prostate specific transglutaminase (pTGase), have been proposed as adjuncts to PSA to increase diagnostic specificity as described, e.g., in Nam et al., J. Clin. Oncol., Vol. 18, pp. 1036-1042 (2000), and reduce the number of men subjected to unnecessary biopsy, but the usefulness of these markers is still under investigation.

A molecular understanding of prostate cancer development and progression is an important step toward the identification of additional biomarkers with increased specificity for invasive prostate cancer and of new tumor-specific therapeutic targets. The benefits of this increased understanding include the recent introduction into clinical trials of immunological reagents based on cell surface antigens, secreted proteins, and intracellular proteins found in prostate cancer as described, e.g., in Saffran et al., Cancer Metastasis Rev., Vol.18, pp. 437-449 (1999). Molecular alterations in prostate cancer include mutations in Rb, p53, PTEN, β-catenin, ras and the androgen receptor but, when present, these occur largely in advanced disease as described, e.g., in Lalani et al., Cancer Metastasis Rev., Vol. 16, pp. 29-66 (1997); Li et al., Science, Vol. 275, pp. 1943-1947 (1997); Marcelli et al., Cancer Res., Vol. 60, pp. 944-949 (2000); and Voeller et al., Cancer Res., Vol. 58, pp. 2520-2523 (1998). Thus, the key molecular alterations that underlie the majority of prostate carcinomas remain unknown.

Analysis of differential gene expression in prostate cancer has been performed primarily by analysis of bulk tissue and cell lines as described, e.g., in Bussemakers et al., Cancer Res., Vol. 59, pp. 5975-5979 (1999); Chang et al., Cancer Res., Vol. 57, pp. 4075-4081 (1997); Hoang et al., Am. J. Pathol., Vol. 156, pp. 857-864 (2000); Huang et al., Genomics, Vol. 59, pp. 178-186 (1999); Pilarsky et al., Prostate, Vol. 36, pp. 85-91 (1998); Sun et al., Cancer Res., Vol. 57, pp. 18-23 (1997); and Yang et al., Cancer Res., Vol. 58, pp. 3732-3735 (1998), but only a few genes from these analyses have been validated in subsequent studies. A concerted effort by the Cancer Genome Anatomy Project using prostate cancer as a model system for gene expression profiling has generated at least 12 prostate cDNA libraries from benign epithelium, PIN and cancer. Epithelial-associated genes were identified among the nearly 30,000 clones sequenced, but most of these were not expressed at sufficient levels to make statistically valid assessments of differential expression between normal and cancer tissue as described, e.g., in Emmert-Buck et al., Am. J. Pathol., Vol. 156, pp. 1109-1115 (2000).

While PSA has proven useful as a diagnostic marker for prostate cancer, there remain deficiencies in its use, in that its production by normal and malignant prostate tissue results in a high false-positive rate for prostate cancer detection. In addition, the above-mentioned prostate specific genes are not typically upregulated in prostate cancer, and thus cannot be utilized to monitor the progression or regression of prostate cancer. Further, the above-mentioned studies have not been completely successful in elucidating key molecular mechanisms involved in prostate cancer. The identification of genes which are differentially expressed in prostate cancer and other prostate disorders and which are linked to the progression of prostate disorders would be of considerable importance not only in the development of a rapid inexpensive method for diagnosing prostate disorders and for providing diagnostic markers specific for prostate disorders but also for monitoring the progression/regression of prostate disorders, elucidating the molecular mechanisms of prostate disorders, and the development of new therapeutic targets for treating men with prostate disorders or at risk of developing prostate disorders.

### SUMMARY OF THE INVENTION

The present invention as described herein below overcomes deficiencies in currently available methods of diagnosing prostate cancer by identifying a plurality of genes that are highly differentially expressed in human prostate disorders, particularly prostate cancer, compared to normal human prostate. The mRNA transcripts and proteins corresponding to these genes have utility, e.g., as diagnostic-markers and therapeutic targets that are specific for prostate cancer and other prostate disorders.

In one aspect, a method for screening a subject for a prostate disorder or at risk of developing a prostate disorder is provided, the method comprising:

a) detecting a level of expression of at least one gene identified in Tables 2, 3 or 4 in a sample of prostate tissue obtained from the subject to provide a first value, with the proviso that if expression of one gene is detected that the gene is not FASN; and

b) comparing the first value with a level of expression of the at least one gene identified in Tables 2, 3 or 4 in a sample of prostate tissue obtained from a disease-free subject, wherein a greater expression level in the subject sample compared to the sample from the disease-free subject is indicative of the subject having a prostate disorder or at risk of developing a prostate disorder.

In another aspect, a method for monitoring the progression of a prostate disorder in a subject having or at risk of having a prostate disorder is provided which comprises measuring a level of expression of at least one gene identified in Tables 2, 3 or 4 over time in a prostate tissue sample obtained from the subject, with the proviso that if expression of only one gene is detected that the gene is not FASN, wherein an increase in the level of expression of the at least one gene over time is indicative of the progression of the prostate disorder in the subject.

In another aspect, a method for identifying agents for use in the treatment of a prostate disorder is provided, the method comprising:

a) contacting a sample of diseased prostate cells with a candidate agent;

b) detecting a level of expression of at least one gene in the diseased prostate cells, wherein the at least one gene is identified in Tables 2, 3 or 4, with the proviso that if expression of only one gene is detected that the gene is not FASN; and

c) comparing the level of expression of the at least one gene in the sample in the presence of the candidate agent with a level of expression of the at least one gene in cells that are not contacted with the candidate agent, wherein a decreased level of expression of the at least one gene in the sample in the presence of the candidate agent relative to the level of expression of the at least one gene in the sample in the absence of the candidate agent is indicative of an agent useful in the treatment of a prostate disorder.

In another aspect, a method of inhibiting undesired proliferation of a prostate cell is provided, wherein the method comprises administering to the cell an effective amount of an agent that can decrease the expression of at least one gene identified in Tables 2, 3 or 4, with the proviso that if expression of only one gene is inhibited that the gene is not FASN.

In another aspect, a method for monitoring the efficacy of a treatment of a subject having a prostate disorder or at risk of developing a prostate disorder with an agent is provided, the method comprising:

a) obtaining a pre-administration sample from the subject prior to administration of the agent,

b) detecting a level of expression of at least one gene identified in Tables 2, 3 or 4 in the pre-administration sample, with the proviso that if expression of only one gene is detected that the gene is not FASN;

c) obtaining one or more post-administration samples from the subject;

d) detecting a level of expression of the at least one gene in the post-administration sample or samples;

e) comparing the level of expression of the at least one gene in the pre-administration sample with the level of expression of the at least one gene in the post-administration sample; and

f) adjusting the administration of the agent accordingly.

In another aspect, a viral vector is provided which comprises a promoter and/or enhancer of at least one gene selected from the group consisting of the genes identified in Tables 2, 3 or 4 with the proviso that the gene is not FASN, operably linked to a coding region of a gene that is essential for replication of the vector, wherein the vector is adapted to replicate upon transfection into a diseased prostate cell.

In another aspect, a nucleic acid construct is provided that comprises a promoter and/or enhancer of at least one gene selected from the group consisting of the genes identified in Tables 2, 3 or 4 with the proviso that the gene is not FASN, operably linked to a coding region of a heterologous gene product.

### BRIEF DESCRIPTION OF THE DRAWINGS

FIG. 1

Dendrogram of 55 experimental samples that are grouped according to overall similarity in level of expression of a subset of 3,530 genes that have varied most across the samples.

FIGS. 2a and 2b

Expression levels of highly ranked genes in normal and malignant prostate tissues. Each gene is represented by two mean values derived from the expression level in 24 malignant (squares) and 9 normal (circles) samples. Error bars represent 99% confidence intervals. Redundant entries are qualified by accession or transcript numbers: a) hybridization signals for 20 genes which were ranked most highly by the “difference” metric; b) genes ranked most highly by the “diagnostic” metric.

FIG. 3

Amplification of hepsin and PLAB transcripts from selected prostate tissues. Transcripts from normal (N8, N10) and tumor (T16, T22) tissues were co-amplified with 18s rRNA. Hybridization intensities of hepsin and PLAB on corresponding microarrays are indicated beneath the PCR product generated from each case.

### DESCRIPTION OF THE INVENTION

All patent applications, patents and literature references cited herein are hereby incorporated by reference in their entirety.

The present invention relates to the identification of genes which are expressed at much higher levels in prostate disorders, particularly cancer, than in normal prostate tissue. By virtue of their low expression in normal body tissues, including normal prostate tissues, and their high expression in neoplastic prostate tissues, these genes can be utilized in the diagnosis, management, treatment, and/or post-treatment follow-up of men at risk for, with, or at risk for recurrence of prostate disorders which include, but are not limited to, localized prostate cancer, metastatic prostate cancer, prostatitis, benign prostatic hypertrophy, and benign prostatic hyperplasia. These 50 genes include those listed in Tables 2, 3 and 4. The complete sequences of the 50 genes are available from UniGene, and TIGR or Entrez Databases using the accession numbers shown in Tables 2, 3 and 4.

Any selection of at least one of the genes listed in Tables 2, 3 or 4 can be utilized as a tumor marker/therapeutic target with the proviso that if expression of only one gene is detected that the gene is not fatty acid synthase (FASN). In some embodiments, if expression of only one gene is detected the gene is not PLAB. In preferred embodiments, any selection of at least one of the 22 genes listed in Tables 2 or 3 can be utilized as a tumor marker/therapeutic target. In particularly useful embodiments, a plurality of these genes, at least two or more of the 50 genes listed in Tables 2, 3 or 4 can be selected and their expression monitored simultaneously to provide expression profiles for use in various aspects. For example, expression profiles of the genes provide valuable molecular tools for rapidly diagnosing and monitoring the progression of prostate disorders, and for evaluating drug efficacy. Changes in the expression profile from a baseline profile can be used as an indication of such effects. Accordingly, the invention provides methods for screening a subject for (diagnostic) a prostate disorder or at risk of developing (prognostic) a prostate disorder, methods for monitoring the progression of a prostate disorder in a subject, methods for the identification of agents that are useful in treating a subject having or at risk of having a prostate disorder, methods of inhibiting undesired proliferation of a prostate cell, methods of treating a subject having or at risk of having a prostate disorder, methods for monitoring the efficacy of certain drug treatments for prostate disorders, and vectors for prostate-specific replication.

Twenty of the most differentially expressed genes are identified in Table 2. Particularly preferred genes listed in Table 2 include hepsin, prostate differentiation factor (PLAB; also known as macrophage inhibitory cytokine, MIC-1; and TGF-beta superfamily protein), alpha-methylacyl-CoA racemase (AMACR) and fatty acid synthase (FASN).

There are several biological features of hepsin, PLAB, AMACR, and FASN that make these genes suitable as diagnostic markers and/or therapeutic targets as described below.

Hepsin is a Type II, membrane-associated serine protease that has been shown to activate Human Factor VII and to initiate a pathway of blood coagulation on the cell surface leading to thrombin formation as described, e.g., in Kazama, J. Biol. Chem., Vol. 270, pp. 66-72 (1995). It is believed that a number of neoplastic cells activate the blood coagulation system, resulting in hypercoagulability and intravascular thrombosis through this and other pathways, and that hepsin plays a role in their cell growth, as described, e.g., in Torres-Rosada et al., Proc. Natl. Acad. Sci. USA, Vol. 90, pp. 7181-7185 (1993). The expression of the hepsin gene is highly restricted; i.e., the gene is lowly expressed in most body tissues, with the exception of high levels in liver and moderate levels in the kidney, as described e.g., in Tsuji et al., J. Biol. Chem., Vol. 266, pp. 16948-16953 (1991). Hepsin has been reported as highly expressed in several cancer cell lines and, most recently, in ovarian cancer, as described, e.g., in Tanimoto et al., Cancer Res., Vol. 57, pp. 2884-2887 (1997). In addition, although expression of hepsin is high in the liver, knockout mice with disruptions in both copies of the hepsin gene do not show liver abnormalities or dysfunction. Indeed, these mice do not show any discernable phenotype, as described, e.g., in Wu et al., J. Clin. Invest., Vol. 101, pp. 321-326 (1998). Antibodies targeted against the extracellular domain of hepsin have been shown to retard the growth of hepatoma cells which over-express hepsin, as described, e.g., in Torres-Rosada et al., supra. Hepsin has also been found to be expressed on the trophoblast surfaces of mouse embryos as described, e.g., in Vu et al., J. Biol. Chem., Vol. 272, pp. 31315-31320 (1997). Data obtained from soluble hepsin expression studies carried out by Vu et al., supra, suggest that expressed one-chain hepsin undergoes autoactivation, and is proteolytically cleaved to generate a two-chain membrane-bound form, wherein the heavy or catalytic chain is linked to the light chain via a disulfide bond. The light chain is attached to the cell surface via a hydrophobic, internal signal sequence. The activated two-chain form of hepsin appears to undergo additional cleavage wherein the catalytic domain of hepsin is cleaved off the trophoblast surfaces of the embryos. Accordingly, the cleaved catalytic domain of hepsin can be detected in the serum, particularly in the serum of subjects having or at risk of developing a prostate disorder such as prostate cancer, wherein hepsin is overexpressed compared with disease-free subjects.

PLAB/MIC-1 was identified by Bootcov et al., Proc. Natl. Acad. Sci. USA, Vol.94, No. 21, pp. 11514-11519 (1997), as a novel macrophage inhibitory cytokine with homology to the TGF-beta superfamily of proteins, and also by Hromos et al., Biochim. Biophys. Acta. Vol. 1354, No. 1, pp. 40-44 (1997), as a novel member of the bone morphogenetic protein family. PLAB/MIC-1 appears to have multiple functions, including inhibition of lipopolysaccharide -induced macrophage TNF-alpha production, which suggests that MIC-1 acts in macrophages as an autocrine regulatory molecule. Its production in response to secreted proinflammatory cytokines and TGF-beta may serve to limit the later phases of macrophage activation. PLAB also inhibits the proliferation of primitive hematopoietic progenitors and high expression of PLAB by placenta raises the possibility that it may be a mediator of placental control of embryonic development. PLAB was later identified as a p53-responsive gene by Kannan et al., FEBS Lett., Vol. 470, No. 1, pp. 77-82 (2000), suggesting a paracrine role for PLAB in p53-mediation growth suppression. PLAB is transcribed as a propeptide, which is cleaved and the peptide is secreted from expressing cells. Accordingly, PLAB is secreted from prostate cancer cells, and is detectable in the blood, and thus, would provide a diagnostic.

The synthesis of fatty acids from acetyl-CoA and malonyl-CoA is carried out by fatty acid synthase, FASN. All of the reactions of fatty acid synthesis are carried out by the multiple enzymatic activities of FASN. Like fat oxidation, fat synthesis involves four enzymatic activities. These are b-keto-ACP synthase (condensing enzyme), b-keto-ACP reductase, 3-OH acyl-ACP dehydratase and enoyl-ACP reductase. The two reduction reactions require NADPH oxidation to NADP⁺. The primary fatty acid synthesized by FASN is palmitate. Palmitate is then released from the enzyme by thioesterase reaction and can then undergo separate elongation and/or unsaturation to yield other fatty acid molecules. FASN has been described as upregulated in aggressive subtypes of several common cancers, e.g., breast, ovary and prostate, as reviewed by Kuhajda, in Nutrition, Vol.16, No. 3, pp. 202-208 (2000).

Alpha-Methylacyl-CoA racemase (AMACR) plays an important role in the beta-oxidation of branched-chain fatty acids and fatty acid derivatives because it catalyzes the conversion of several (2R)-methyl-branched-chain fatty acyl-CoAs to their (S)-stereoisomers. Only stereoisomers with the 2-methyl group in the (S)-configuration can be degraded via beta-oxidation (described in Ferdenandusse et al., J. Lipid Res., Vol. 41, No. 11, pp. 1890-1896 (2000). AMACR over-expression in tumor cells has not been described to date.

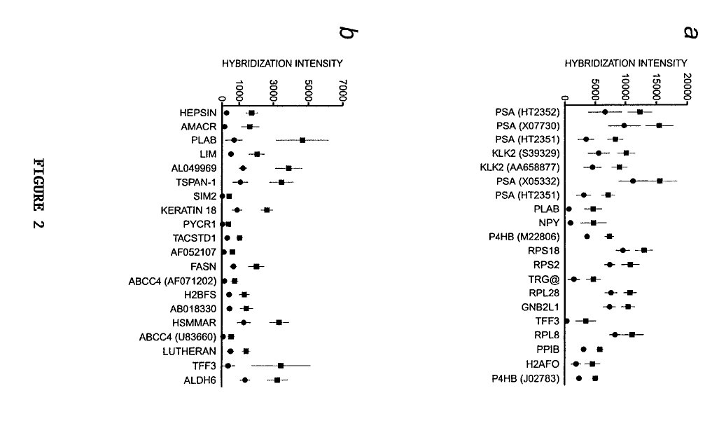

Two other genes that are differentially expressed in normal and malignant prostate tissues are alternative splice forms of prostate specific antigen identified in Tables 3 and 4 and FIG. 2A known as prostate specific antigen, alternative splice form 2 (accession number HT2351) and prostate specific antigen, alternative splice form 3 (accession number HT2352). Both of these alternative splice forms show high expression in prostate cancer tissue and low expression in normal prostate tissue and other body tissues. The complete sequences of the splice forms of the PSA genes are available from TIGR Database using the disclosed accession numbers. Table 4 includes the genes listed in Tables 2 and 3 and also includes other genes that are differentially expressed in normal and malignant prostate tissues. The sequences of the genes in UniGene, TIGR and Entrez are expressly incorporated herein by reference.

The method for screening a subject for a prostate disorder or at risk of developing a prostate disorder comprises:

a) detecting a level of expression of at least one of the genes identified in Tables 2, 3 or 4 in a sample of bodily fluid or prostate tissue obtained from the subject to provide a first value; and

b) comparing the first value with a level of expression of the at least one gene identified in Tables 2, 3 or 4 in a sample of prostate tissue obtained from a disease-free subject, wherein a greater or lesser, particularly greater, expression level in the subject sample, compared to the sample from the disease-free subject, is indicative of the subject having a prostate disorder or at risk of developing a prostate disorder.

In one embodiment, if expression of only one gene is detected the gene is not FASN. In another embodiment, if expression of only one gene is detected the gene is not PLAB.

The prostate tissue sample can be obtained from the subject, a human or animal model, by known surgical methods, e.g., surgical resection or needle biopsy. The sample of bodily fluid, preferably blood, from the subject can also be obtained by standard methods. When assessing the level of expression of the gene by measuring the level of mRNA as described below, it is preferable to obtain a prostate tissue sample from the subject. The sample taken from the disease-free subject can be a sample of normal prostate tissue or bodily fluid from the same individual or from another individual. For example, in examination of a suspected prostate disorder such as prostate cancer, the sample from the disease-free subject can be a sample of normal prostate cells from the individual suspected of having prostate cancer. These normal prostate cells can be obtained from a site adjacent to the tissue suspected of containing prostate cells. Alternatively, the sample taken from the disease-free subject can be a sample of normal prostate cells or bodily fluid obtained from another individual. The sample obtained from the disease-free subject can be obtained at the same time as the sample obtained from the subject, or can be a pre-established control for which expression of the gene was determined at an earlier time. The level of expression of the gene in the sample obtained from the disease-free subject is determined and quantitated using the same approach as used for the sample obtained from the subject.

The level of expression of at least one of the disclosed genes in the samples obtained from the subject and disease-free subject can be detected by measuring either the level of mRNA corresponding to the gene, the protein encoded by the gene or a fragment of the protein, e.g., the catalytic domain of hepsin. In the methods of the invention, the level of expression of one of the disclosed genes in a diseased prostate tissue preferably differs from the level of expression of the gene in a non-diseased tissue by a statistically significant amount. In presently preferred embodiments, at least about a 2-fold difference in expression levels is observed. In some embodiments, the expression levels of a gene differ by at least about 5-, 10- or 100-fold or more in the diseased tissue compared to the non-diseased tissue.

The level of expression of at least one of the genes disclosed in Tables 2, 3 or 4 is determined in the methods of the invention. It is sometimes desirable to determine the level of expression of 2, 3, 5, 10, 20, or more of the disclosed genes.

RNA can be isolated from the samples by methods well known to those skilled in the art as described, e.g., in Ausubel et al., Current Protocols in Molecular Biology, Vol. 1, pp. 4.1.1-4.2.9 and 4.5.1-4.5.3, John Wiley & Sons, Inc. (1996). Methods for detecting the level of expression of mRNA are well known in the art and include, but are not limited to, northern blotting, reverse transcription PCR, real time quantitative PCR and other hybridization methods. A particularly useful method for detecting the level of mRNA transcripts obtained from a plurality of the disclosed genes involves hybridization of labeled mRNA to an ordered array of oligonucleotides. Such a method allows the level of transcription of a plurality of these genes to be determined simultaneously to generate gene expression profiles or patterns. The gene expression profile derived from the sample obtained from the subject can be compared with the gene expression profile derived from the sample obtained from the disease-free subject to determine whether the genes are over-expressed in the sample from the subject relative to the genes in the sample obtained from the disease-free subject, and thereby determine whether the subject has or is at risk of developing a prostate disorder.

The oligonucleotides utilized in this hybridization method typically are bound to a solid support. Examples of solid supports include, but are not limited to, membranes, filters, slides, paper, nylon, wafers, fibers, magnetic or nonmagnetic beads, gels, tubing, polymers, polyvinyl chloride dishes, etc. Any solid surface to which the oligonucleotides can be bound, either directly or indirectly, either covalently or non-covalently, can be used. A particularly preferred solid substrate is a high density array or DNA chip (see Materials and Methods and Example 1). These high density arrays contain a particular oligonucleotide probe in a pre-selected location on the array. Each pre-selected location can contain more than one molecule of the particular probe. Because the oligonucleotides are at specified locations on the substrate, the hybridization patterns and intensities (which together result in a unique expression profile or pattern) can be interpreted in terms of expression levels of particular genes.

The oligonucleotide probes are preferably of sufficient length to specifically hybridize only to complementary transcripts of the above identified gene(s) of interest. As used herein, the term “oligonucleotide” refers to a single-stranded nucleic acid. Generally the oligonucleotides probes will be at least 16 to 20 nucleotides in length, although in some cases longer probes of at least 20 to 25 nucleotides will be desirable.

The oligonucleotide probes can be labeled with one or more labeling moieties to permit detection of the hybridized probe/target polynucleotide complexes. Labeling moieties can include compositions that can be detected by spectroscopic, biochemical, photochemical, bioelectronic, immunochemical, electrical optical or chemical means. Examples of labeling moieties include, but are not limited to, radioisotopes, e.g., ³²P, ³³P, ³⁵S, chemiluminescent compounds, labeled binding proteins, heavy metal atoms, spectroscopic markers such as fluorescent markers and dyes, linked enzymes, mass spectrometry tags, and magnetic labels.

Oligonucleotide probe arrays for expression monitoring can be prepared and used according to techniques which are well known to those skilled in the art as described, e.g., in Lockhart et al., Nature Biotechnology, Vol. 14, pp. 1675-1680 (1996); McGall et al., Proc. Natl. Acad. Sci. USA, Vol. 93, pp. 13555-13460 (1996); and U.S. Pat. No. 6,040,138.

Expression of the protein encoded by the gene(s) or a fragment of the protein, e.g., the catalytic domain of hepsin, can be detected by a probe which is detectably labeled, or which can be subsequently labeled. Generally, the probe is an antibody which recognizes the expressed protein.

As used herein, the term antibody includes, but is not limited to, polyclonal antibodies, monoclonal antibodies, humanized or chimeric antibodies and biologically functional antibody fragments, which are those fragments sufficient for binding of the antibody fragment to the protein or a fragment of the protein.

For the production of antibodies to a protein encoded by one of the disclosed genes or to a fragment of the protein, various host animals may be immunized by injection with the polypeptide, or a portion thereof. Such host animals may include, but are not limited to, rabbits, mice and rats, to name but a few. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler and Milstein (Nature, Vol. 256, pp. 495-497 (1975); and U.S. Pat. No. 4,376,110), the human B-cell hybridoma technique (Kosbor et al., Immunology Today, Vol. 4, p. 72 (1983); Cole et al., Proc. Natl. Acad. Sci. USA, Vol. 80, pp. 2026-2030 (1983)), and the EBV-hybridoma technique (Cole et al., Monoclonal Antibodies and Cancer Therapy, Alan R. Liss, Inc., pp. 77-96 (1985)). Such antibodies may be of any immunoglobulin class, including IgG, lgM, IgE, IgA, IgQ and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., Proc. Nati. Acad. Sci. USA, Vol. 81, pp. 6851-6855 (1984); Neuberger et al., Nature, Vol. 312, pp. 604-608 (1984); Takeda et al., Nature, Vol. 314, pp. 452-454 (1985)) by splicing the genes from a mouse antibody molecule of appropriate antigen specificity, together with genes from a human antibody molecule of appropriate biological activity, can be used. A chimeric antibody is a molecule in which different portions are derived from different animal species, such as those having a variable or hypervariable region derived from a murine mAb and a human immunoglobulin constant region.

Alternatively, techniques described for the production of single-chain antibodies (U.S. Pat. No. 4,946,778; Bird, Science, Vol. 242, pp. 423-426 (1988); Huston et al., Proc. Natl. Acad. Sci. USA, Vol. 85, pp. 5879-5883 (1988); and Ward et al., Nature, Vol. 334, pp. 544-546 (1989)) can be adapted to produce differentially expressed gene-single chain antibodies. Single chain antibodies are formed by linking the heavy and light chain fragments of the Fv region via an amino acid bridge, resulting in a single-chain polypeptide.

Most preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al., Science, Vol. 246, pp. 1275-1281 (1989)) to allow rapid and easy identification of monoclonal Fab fragments with the desired specificity.

The extent to which the known proteins are expressed in the sample is then determined by immunoassay methods which utilize the antibodies described above. Such immunoassay methods include, but are not limited to, dot blotting, western blotting, competitive and noncompetitive protein binding assays, enzyme-linked immunosorbant assays (ELISA), immunohistochemistry, fluorescence-activated cell sorting (FACS), and others commonly used and widely described in scientific and patent literature, and many employed commercially.

Particularly preferred, for ease of detection, is the sandwich ELISA, of which a number of variations exist, all of which are intended to be encompassed by the present invention. For example, in a typical forward assay, unlabeled antibody is immobilized on a solid substrate and the sample to be tested is brought into contact with the bound molecule and incubated for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay, in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the protein expressed by the gene of interest, e.g., PLAB, hepsin or a fragment thereof.

The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate. As will be readily recognized, however, a wide variety of different ligation techniques exist which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product, rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of secreted protein or fragment thereof, e.g., PLAB or the catalytic domain of hepsin, which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

In another aspect, kits are provided for detecting the level of expression of at least one gene identified in Tables 2, 3 or 4 in a biological sample, e.g., a sample of bodily fluid or prostate tissue, with the proviso that if expression of only one gene is detected that the gene is not FASN. In some embodiments, if expression of only one gene is detected the gene is not PLAB. For example, the kit can comprise a labeled compound or agent capable of detecting a protein encoded by, or mRNA corresponding to, at least one of the genes identified in Tables 2, 3 or 4; or fragment of the protein, means for determining the amount of protein encoded by or mRNA corresponding to the gene or fragment of the protein; and means for comparing the amount of protein encoded by or mRNA corresponding to the gene or fragment of the protein, obtained from the subject sample with a standard level of expression of the gene, e.g., from a disease-free subject. The compound or agent can be packaged in a suitable container. The kit can further comprise instructions for using the kit to detect protein encoded by or mRNA corresponding to the gene.

In another aspect, progression of a prostate disorder in a subject can be monitored by measuring a level of expression of mRNA corresponding to, or protein encoded by, at least one of the genes identified in Tables 2, 3 or 4, in a sample of bodily fluid or prostate tissue obtained in the subject over time, i.e., at various stages of the prostate disorder, with the proviso that if expression of only one gene is detected that the gene is not FASN. In some embodiments, if expression of only one gene is detected the gene is not PLAB. An increase in the level of expression of the mRNA or encoded protein corresponding to the gene(s) over time is indicative of the progression of the prostate disorder. The level of expression of mRNA and protein corresponding to the gene(s) can be detected by standard methods as described above.

In a particularly useful embodiment, the level of mRNA expression of a plurality of the disclosed genes, at least two of the 50 genes, and more preferably two of the 22 genes listed in Tables 2 and 3, can be measured simultaneously in a subject at various stages of the prostate disorder to generate a transcriptional or expression profile of the prostate disorder over time. For example, mRNA transcripts corresponding to a plurality of these genes can be obtained from prostate cells of a subject at different times, and hybridized to a chip containing oligonucleotide probes which are complementary to the transcripts of the desired genes, to compare expression of a large number of genes at various stages of the prostate disorder.

In another aspect, a cell-based assay based on the disclosed genes can be used to identify agents that can modulate the expression of one or more genes that are differentially expressed in diseased prostate cells compared to non-diseased prostate cells. Such agents are suitable for use in the treatment of a prostate disorder, and are useful in studies of the morphogenesis and progression of prostate disease. These methods generally involve comparing the expression level of one or more of the disclosed genes in a prostate cell obtained from a diseased tissue that is contacted with a candidate agent to the expression level of the gene or genes in cells that are not contacted with the candidate agent. Cells and cell lines that are suitable for use in these assays are publicly available. For example, numerous cell lines obtained from diseased prostate tissue are available from the American Type Culture Collection (10801 University Boulevard, Manassas, Va. 20110-2209; www.ATCC.org). One can also obtain suitable cells directly or indirectly from a subject that has a prostate disorder. Thus, in some embodiments, the method comprises:

a) contacting a sample of diseased prostate cells with a candidate agent;

b) detecting a level of expression of at least one gene identified in Tables 2, 3 or 4 in the sample of diseased prostate cells; and

c) comparing the level of expression of the gene in the sample in the presence of the candidate agent with a level of expression of the gene in the sample that is not contacted with the candidate agent, wherein a decreased level of expression in the sample in the presence of the agent relative to the level of expression in the absence of the agent is indicative of an agent useful in the treatment of a prostate disorder.

In some embodiments, if expression of only one gene is detected the gene is not FASN. In other embodiments, if expression of only one gene is detected the gene is not PLAB. The level of expression of the gene is detected by measuring the level of mRNA corresponding to, or protein encoded by, the gene as described above.

As used herein, the term “candidate agent” refers to any molecule that is capable of decreasing the level of mRNA corresponding to, or protein encoded by, at least one of the disclosed genes. The candidate agent can be natural or synthetic molecules such as proteins or fragments thereof, small molecule inhibitors, nucleic acid molecules, e.g., antisense nucleotides, ribozymes, double-stranded RNAs, organic and inorganic compounds and the like.

Cell-free assays can also be used to identify compounds which are capable of interacting with a protein encoded by one of the disclosed genes or protein-binding partner to alter the activity of the protein or its binding partner. Cell-free assays can also be used to identify compounds which modulate the interaction between the encoded protein and its binding partner such as a target peptide. In one embodiment, cell-free assays for identifying such compounds comprise a reaction mixture containing a protein encoded by one of the disclosed genes and a test compound or a library of test compounds in the presence or absence of the binding partner, e.g., a biologically inactive target peptide, or a small molecule. Accordingly, one example of a cell-free method for identifying agents useful in the treatment of prostate disorders is provided which comprises contacting a protein or functional fragment thereof or the protein binding partner with a test compound or library of test compounds and detecting the formation of complexes. For detection purposes, the protein can be labeled with a specific marker and the test compound or library of test compounds labeled with a different marker. Interaction of a test compound with the protein or fragment thereof or the protein-binding partner can then be detected by measuring the level of the two labels after incubation and washing steps. The presence of the two labels is indicative of an interaction.

Interaction between molecules can also be assessed by using real-time BIA (Biomolecular Interaction Analysis, Pharmacia Biosensor AB), which detects surface plasmon resonance, an optical phenomenon. Detection depends on changes in the mass concentration of mass macromolecules at the biospecific interface and does not require labeling of the molecules. In one useful embodiment, a library of test compounds can be immobilized on a sensor surface, e.g., a wall of a micro-flow cell. A solution containing the protein, functional fragment thereof, or the protein-binding partner is then continuously circulated over the sensor surface. An alteration in the resonance angle, as indicated on a signal recording, indicates the occurrence of an interaction. This technique is described in more detail in BlAtechnology Handbook by Pharmacia.

Another embodiment of a cell-free assay comprises:

a) combining a protein encoded by the at least one gene, the protein binding partner, and a test compound to form a reaction mixture and

b) detecting interaction of the protein and the protein binding partner in the presence and absence of the test compounds.

A considerable change (potentiation or inhibition) in the interaction of the protein and binding partner in the presence of the test compound compared to the interaction in the absence of the test compound indicates a potential agonist (mimetic or potentiator) or antagonist (inhibitor) of the proteins activity for the test compound. The components of the assay can be combined simultaneously or the protein can be contacted with the test compound for a period of time, followed by the addition of the binding partner to the reaction mixture. The efficacy of the compound can be assessed by using various concentrations of the compound to generate dose response curves. A control assay can also be performed by quantitating the formation of the complex between the protein and its binding partner in the absence of the test compound.

Formation of a complex between the protein and its binding partner can be detected by using detectably labeled proteins such as radiolabeled, fluorescently labeled, or enzymatically labeled protein or its binding partner, by immunoassay or by chromatographic detection.

In preferred embodiments, the protein or its binding partner can be immobilized to facilitate separation of complexes from uncomplexed forms of the protein and its binding partner and automation of the assay. Complexation of the protein to its binding partner can be achieved in any type of vessel, e.g., microtitre plates, micro-centrifuge tubes and test tubes. In particularly preferred embodiments, the protein can be fused to another protein, e.g., glutathione-S-transferase to form a fusion protein which can be adsorbed onto a matrix, e.g., glutathione sepharose beads (Sigma Chemical. St. Louis, Mo.), which are then combined with the labeled protein partner, e.g., labeled with ³⁵S, and test compound and incubated under conditions sufficient to formation of complexes. Subsequently, the beads are washed to remove unbound label, and the matrix is immobilized and the radiolabel is determined. The aforementioned cell-free assays are particularly useful with proteins encoded by the genes hepsin, FASN, and MOAT-B.

Another method for immobilizing proteins on matrices involves utilizing biotin and streptavidin. For example, the protein can be biotinylated using biotin NHS (N-hydroxy-succinimide), using well known techniques and immobilized in the well of streptavidin-coated plates.

Cell-free assays can also be used to identify agents which are capable of interacting with a protein encoded by the at least one gene and modulate the activity of the protein encoded by the gene. In one embodiment, the protein is incubated with a test compound and the catalytic activity of the protein is determined. In another embodiment, the binding affinity of the protein to a target molecule can be determined by methods known in the art.

The present invention also provides for both prophylactic and therapeutic methods of treating a subject having, or at risk of having, a prostate disorder. Subjects at risk for such disorders can be identified by a prognostic assay, e.g., as described above. Administration of a prophylactic agent can occur prior to the manifestation of symptoms characteristic of the prostate disorder, such that development of the prostate disorder is prevented or delayed in its progression. With respect to treatment of the prostate disorder, it is not required that the prostate cell, e.g., cancer cell, be killed or induced to undergo cell death. Instead, all that is required to achieve treatment of the prostate disorder is that the tumor growth be slowed down to some degree or that some of the abnormal cells revert back to normal. Examples of suitable therapeutic agents include, but are not limited to, antisense nucleotides, ribozymes, double-stranded RNAs and antagonists as described in detail below.

As used herein, the term “antisense” refers to nucleotide sequences that are complementary to a portion of an RNA expression product of at least one of the disclosed genes. “Complementary” nucleotide sequences refer to nucleotide sequences that are capable of base-pairing according to the standard Watson-Crick complementarity rules. That is, purines will base- pair with pyrimidine to form combinations of guanine:cytosine and adenine:thymine in the case of DNA, or adenine:uracil in the case of RNA. Other less common bases, e.g., inosine, 5-methylcytosine, 6-methyladenine, hypoxanthine and others may be included in the hybridizing sequences and will not interfere with pairing.

When introduced into a host cell, antisense nucleotide sequences specifically hybridize with the cellular mRNA and/or genomic DNA corresponding to the gene(s) so as to inhibit expression of the encoded protein, e.g., by inhibiting transcription and/or translation within the cell.

The isolated nucleic acid molecule comprising the antisense nucleotide sequence can be delivered, e.g., as an expression vector, which when transcribed in the cell, produces RNA which is complementary to at least a unique portion of the encoded mRNA of the gene(s). Alternatively, the isolated nucleic acid molecule comprising the antisense nucleotide sequence is an oligonucleotide probe which is prepared ex vivo and, which, when introduced into the cell, results in inhibiting expression of the encoded protein by hybridizing with the mRNA and/or genomic sequences of the gene(s).

Preferably, the oligonucleotide contains artificial internucleotide linkages which render the antisense molecule resistant to exonucleases and endonucleases, and thus are stable in the cell. Examples of modified nucleic acid molecules for use as antisense nucleotide sequences are phosphoramidate, phosporothioate and methylphosphonate analogs of DNA as described, e.g., in U.S. Pat. Nos. 5,176,996; 5,264,564; and 5,256,775. General approaches to preparing oligomers useful in antisense therapy are described, e.g., in Van der Krol, BioTechniques, Vol. 6, pp. 958-976 (1988); and Stein et al., Cancer Res., Vol. 48, pp. 2659-2668 (1988).

Typical antisense approaches, involve the preparation of oligonucleotides, either DNA or RNA, that are complementary to the encoded mRNA of the gene. The antisense oligonucleotides will hybridize to the encoded mRNA of the gene and prevent translation. The capacity of the antisense nucleotide sequence to hybridize with the desired gene will depend on the degree of complementarity and the length of the antisense nucleotide sequence. Typically, as the length of the hybridizing nucleic acid increases, the more base mismatches with an RNA it may contain and still form a stable duplex or triplex. One skilled in the art can determine a tolerable degree of mismatch by use of conventional procedures to determine the melting point of the hybridized complexes.

Antisense oligonucleotides are preferably designed to be complementary to the 5′ end of the mRNA, e.g., the 5′ untranslated sequence up to and including the regions complementary to the mRNA initiation site, i.e., AUG. However, oligonucleotide sequences that are complementary to the 3′ untranslated sequence of mRNA have also been shown to be effective at inhibiting translation of mRNAs as described, e.g., in Wagner, Nature, Vol. 372, pp. 333 (1994). While antisense oligonucleotides can be designed to be complementary to the mRNA coding regions, such oligonucleotides are less efficient inhibitors of translation.

Regardless of the mRNA region to which they hybridize, antisense oligonucleotides are generally from about 15 to about 25 nucleotides in length.

The antisense nucleotide can also comprise at least one modified base moiety, e.g., 3-methylcytosine, 5,-methylcytosine, 7-methylguanine, 5-fluorouracil, 5-bromouracil, and may also comprise at least one modified sugar moiety, e.g., arabinose, hexose, 2-fluorarabinose, and xylulose.

In another embodiment, the antisense nucleotide sequence is an alpha-anomeric nucleotide sequence. An alpha-anomeric nucleotide sequence forms specific double stranded hybrids with complementary RNA, in which, contrary to the usual beta-units, the strands run parallel to each other as described e.g., in Gautier et al., Nucl. Acids. Res., Vol. 15, pp. 6625-6641 (1987).

Antisense nucleotides can be delivered to cells which express the described genes in vivo by various techniques, e.g., injection directly into the prostate tissue site, entrapping the antisense nucleotide in a liposome, by administering modified antisense nucleotides which are targeted to the prostate cells by linking the antisense nucleotides to peptides or antibodies that specifically bind receptors or antigens expressed on the cell surface.

However, with the above-mentioned delivery methods, it may be difficult to attain intracellular concentrations sufficient to inhibit translation of endogenous mRNA. Accordingly, in a preferred embodiment, the nucleic acid comprising an antisense nucleotide sequence is placed under the transcriptional control of a promoter, i.e., a DNA sequence which is required to initiate transcription of the specific genes, to form an expression construct. The use of such a construct to transfect cells results in the transcription of sufficient amounts of single stranded RNAs to hybridize with the endogenous mRNAs of the described genes, thereby inhibiting translation of the encoded mRNA of the gene. For example, a vector can be introduced in vivo such that it is taken up by a cell and directs the transcription of the antisense nucleotide sequence. Such vectors can be constructed by standard recombinant technology methods. Typical expression vectors include bacterial plasmids or phage, such as those of the pUC or Bluescript.TM plasmid series, or viral vectors such as adenovirus, adeno-associated virus, herpes virus, vaccinia virus and retrovirus adapted for use in eukaryotic cells. Expression of the antisense nucleotide sequence can be achieved by any promoter known in the art to act in mammalian cells. Examples of such promoters include, but are not limited to, the promoter contained in the 3′ long terminal repeat of Rous sarcoma virsu as described, e.g., in Yamamoto et al., Cell, Vol. 22, pp. 787-797 (1980); the herpes thymidine kinase promoter as described, e.g., in Wagner et al., Proc. Natl. Acad. Sci. USA, Vol. 78, pp. 1441-1445 (1981); the SV40 early promoter region as described, e.g., in Bernoist and Chambon, Nature, Vol. 290, pp. 304-310 (1981); and the regulatory sequences of the metallothionein gene as described, e.g., in Brinster et al., Nature, Vol. 296, pp. 39-42 (1982).

Ribozymes are RNA molecules that specifically cleave other single-stranded RNA in a manner similar to DNA restriction endonucleases. By modifying the nucleotide sequences encoding the RNAs, ribozymes can be synthesized to recognize specific nucleotide sequences in a molecule and cleave it as described, e.g., in Cech, J. Amer. Med. Assn., Vol. 260, p. 3030 (1988). Accordingly, only mRNAs with specific sequences are cleaved and inactivated.

Two basic types of ribozymes include the “hammerhead”-type as described for example in Rossie et al., Pharmac. Ther., Vol. 50, pp. 245-254 (1991); and the hairpin ribozyme as described, e.g., in Hampel et al., Nucl. Acids Res., Vol. 18, pp. 299-304 (1999) and U.S. Pat. No. 5,254,678. Intracellular expression of hammerhead and hairpin ribozymes targeted to mRNA corresponding to at least one of the disclosed genes can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozyme sequences can be modified in essentially the same manner as described for antisense nucleotides, e.g., the ribozyme sequence can comprise a modified base moiety.

Double-stranded RNA, i.e., sense-antisense RNA, corresponding to at least one of the disclosed genes, can also be utilized to interfere with expression of at least one of the disclosed genes. Interference with the function and expression of endogenous genes by double-stranded RNA has been shown in various organisms such as C. elegans as described, e.g., in Fire et al., Nature, Vol. 391, pp.806-811 (1998); drosophilia as described, e.g., in Kennerdell et al., Cell, Vol. 95, No. 7, pp. 1017-1026 (1998); and mouse embryos as described, e.g., in Wianni et al., Nat. Cell Biol., Vol. 2, No. 2, pp. 70-75 (2000). Such double-stranded RNA can be synthesized by in vitro transcription of single-stranded RNA read from both directions of a template and in vitro annealing of sense and antisense RNA strands. Double-stranded RNA can also be synthesized from a cDNA vector construct in which the gene of interest is cloned in opposing orientations separated by an inverted repeat. Following cell transfection, the RNA is transcribed and the complementary strands reanneal. Double-stranded RNA corresponding to at least one of the disclosed genes could be introduced into a prostate cell by cell transfection of a construct such as that described above.

The term “antagonist” refers to a molecule which, when bound to the protein encoded by the gene, inhibits its activity. Antagonists can include, but are not limited to, peptides, proteins, carbohydrates, and small molecules.

In a particularly useful embodiment, the antagonist is an antibody specific for the cell-surface protein expressed by the at least one gene, e.g., hepsin, LIM, NET-1, GA733-2, MOAT-B, MRP4, and Lutheran. Antibodies useful as therapeutics encompass the antibodies as described above. The antibody alone may act as an effector of therapy or it may recruit other cells to actually effect cell killing. The antibody may also be conjugated to a reagent such as a chemotherapeutic, radionuclide, ricin A chain, cholera toxin, pertussis toxin, etc., and serve as a target agent. Alternatively, the effector may be a lymphocyte carrying a surface molecule that interacts, either directly or indirectly, with a tumor target. Various effector cells include cytotoxic T cells and NK cells.

Examples of the antibody-therapeutic agent conjugates which can be used in therapy include, but are not limited to: 1) antibodies coupled to radionuclides, such as ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re, and as described, e.g., in Goldenberg et al., Cancer Res., Vol. 41, pp. 4354-4360 (1981); Carrasquillo et al., Cancer Treat. Rep., Vol. 68, pp. 317-328 (1984); Zalcberg et al., J. NatI. Cancer Inst., Vol. 72, pp. 697-704 (1984); Jones et al., Int. J. Cancer, Vol. 35, pp. 715-720 (1985); Lange et al., Surgery, Vol. 98, pp. 143-150 (1985); Kaltovich et al., J. Nucl. Med., Vol. 27, p. 897 (1986); Order et al., Int. J. Radiother. Oncol. Biol. Phys., Vol. 8, pp. 259-261 (1982); Courtenay-Luck et al., Lancet, Vol. 1, pp. 1441-1443 (1984); and Ettinger et al., Cancer Treat. Rep., Vol. 66, pp. 289-297 (1982); (2) antibodies coupled to drugs or biological response modifiers such as methotrexate, adriamycin, and lymphokines such as interferon as described, for, e.g., in Chabner et al., Cancer, Principles and Practice of Oncology, Philadelphia, Pa., J. B. Lippincott Co. Vol. 1, pp. 290-328 (1985); Oldham et al., Cancer, Principles and Practice of Oncology, Philadelphia, Pa., J. B. Lippincott Co., Vol. 2, pp. 2223-2245 (1985); Deguchi et al., Cancer Res., Vol. 46, pp. 3751-3755 (1986); Deguchi et al., Fed. Proc., Vol. 44, p. 1684 (1985); Embleton et al., Br. J. Cancer, Vol. 49, pp. 559-565 (1984); and Pimm et al., Cancer Immunol. Immunother., Vol.12, pp. 125-134 (1982); (3) antibodies coupled to toxins, as described, for example, in Uhr et al., Monoclonal Antibodies and Cancer, Academic Press, Inc., pp. 85-98 (1983); Vitetta et al., Biotechnology and Bio. Frontiers, Ed. P. H. Abelson, pp. 73-85 (1984); and Vitetta et al., Science, Vol. 219, pp. 644-650 (1983); (4) heterofunctional antibodies, for example, antibodies coupled or combined with another antibody so that the complex binds both to the carcinoma and effector cells, e.g., killer cells such as T cells, as described, for example, in Perez et al., J. Exper. Med., Vol. 163, pp. 166-178 (1986); and Lau et al., Proc. Natl. Acad. Sci. USA, Vol. 82, pp. 8648-8652 (1985); and (5) native, i.e., non-conjugated or non-complexed antibodies, as described in, for example, Herlyn et al., Proc. Natl. Acad. Sci. USA, Vol. 79, pp. 4761-4765 (1982); Schulz et al., Proc. Natl. Acad. Sci. USA, Vol. 80, pp. 5407-5411 (1983); Capone et al., Proc. Natl. Acad. Sci. USA, Vol. 80, pp. 7328-7332 (1983); Sears et al., Cancer Res., Vol. 45, pp. 5910-5913 (1985); Nepom et al., Proc. NatI. Acad. Sci. USA, Vol. 81, pp. 2864-2867 (1984); Koprowski et al., Proc. Natl. Acad. Sci. USA, Vol. 81, pp. 216-219 (1984); and Houghton et al., Proc. Natl. Acad. Sci. USA, Vol. 82, pp. 1242-1246 (1985).

Methods for coupling an antibody or fragment thereof to a therapeutic agent as described above are well known in the art and are described, e.g., in the methods provided in the references above. In yet another embodiment, the antagonist useful as a therapeutic for treating prostate disorders can be an inhibitor of a protein encoded by one of the disclosed genes. For example, the activity of the membrane-bound serine protease hepsin can be inhibited by utilizing specific serine protease inhibitors, which in turn would block the growth of malignant prostate cells with minimal systemic toxicity. Such serine-protease inhibitors are well known in the art as described, e.g., in Leung et al., “Protease Inhibitors: Current Status and Future Prospects”, J. Med. Chem., Vol. 43, pp. 305-341 (2000).

In the case of treatment with an antisense nucleotide, the method comprises administering a therapeutically effective amount of an isolated nucleic acid molecule comprising an antisense nucleotide sequence derived from at least one gene identified in Tables 2, 3 or 4, with the proviso that if expression of only one gene is inhibited that the gene is not FASN, wherein the antisense nucleotide has the ability to decrease the transcription/translation of the at least one gene. In some embodiments, if expression of only one gene is inhibited the gene is not PLAB. The term “isolated” nucleic acid molecule means that the nucleic acid molecule is removed from its original environment (e.g., the natural environment if it is naturally occurring). For example, a naturally occurring nucleic acid molecule is not isolated, but the same nucleic acid molecule, separated from some or all of the co-existing materials in the natural system, is isolated, even if subsequently reintroduced into the natural system. Such nucleic acid molecules could be part of a vector or part of a composition and still be isolated, in that such vector or composition is not part of its natural environment.

With respect to treatment with a ribozyme or double-stranded RNA molecule, the method comprises administering a therapeutically effective amount of a nucleotide sequence encoding a ribozyme, or a double-stranded RNA molecule, wherein the nucleotide sequence encoding the ribozyme/double-stranded RNA molecule has the ability to decrease the transcription/translation of at least one gene identified in Tables 2, 3 or 4, with the proviso that if expression of only one gene is inhibited that the gene is not FASN. In another embodiment, if expression of only one gene is inhibited the gene is not PLAB.

In the case of treatment with an antagonist, the method comprises administering to a subject a therapeutically effective amount of an antagonist that inhibits a protein encoded by at least one gene identified in Tables 2, 3 or 4, with the proviso that if expression of only one gene is inhibited that the gene is not FASN. In another embodiment, if expression of only one gene is inhibited the gene is not PLAB.

A “therapeutically effective amount” of an isolated nucleic acid molecule comprising an antisense nucleotide, nucleotide sequence encoding a ribozyme, double-stranded RNA, or antagonist, refers to a sufficient amount of one of these therapeutic agents to treat a prostate disorder (e.g., to limit prostate tumor growth or to slow or block tumor metastasis). The determination of a therapeutically effective amount is well within the capability of those skilled in the art. For any therapeutic, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Antisense nucleotides, ribozymes, double-stranded RNAs, and antagonists which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range, depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for antagonists.

For therapeutic applications, the antisense nucleotides, nucleotide sequences encoding ribozymes, double-stranded RNAs (whether entrapped in a liposome or contained in a viral vector) and antibodies are preferably administered as pharmaceutical compositions containing the therapeutic agent in combination with one or more pharmaceutically acceptable carriers. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The pharmaceutical compositions may be administered by any number of routes, including, but not limited to, oral, intravenous, intramuscular, intra-articular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated from aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration of the antisense nucleotide or antagonist, such labeling would include amount, frequency, and method of administration. Those skilled in the art will employ different formulations for antisense nucleotides than for antagonists, e.g., antibodies or inhibitors. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

In another aspect, the treatment of a subject with a therapeutic agent, such as those described above, can be monitored by detecting the level of expression of mRNA or protein encoded by at least one of the disclosed genes identified in Tables 2, 3 or 4, or the activity of the protein encoded by the at least one gene. These measurements will indicate whether the treatment is effective or whether it should be adjusted or optimized. Accordingly, one or more of the genes described herein can be used as a marker for the efficacy of a drug during clinical trials.

In a particularly useful embodiment, a method for monitoring the efficacy of a treatment of a subject having a prostate disorder. or at risk of. or having a prostate disorder with an agent (e.g., an antagonist, protein, nucleic acid, small molecule, or other therapeutic agent or candidate agent identified by the screening assays described herein) is provided comprising:

a) obtaining a pre-administration sample from a subject prior to administration of the agent,

b) detecting the level of expression of mRNA corresponding to, or protein encoded by the at least one gene, or activity of the protein encoded by the at least one gene identified in Tables 2, 3 or 4 in the pre-administration sample;

c) obtaining one or more post-administration samples from the subject,

d) detecting the level of expression of mRNA corresponding to, or protein encoded by the at least one gene, or activity of the protein encoded by the at least one gene in the post-administration sample or samples,

e) comparing the level of expression of mRNA or protein encoded by the at least one gene, or activity of the protein encoded by the at least one gene in the pre-administration sample with the level of expression of mRNA or protein encoded by the at least one gene, or activity of the protein encoded by the at least one gene in the post-administration sample or samples, and

f) adjusting the administration of the agent accordingly.

In some embodiments, if expression corresponding to, or activity of protein encoded by, only one gene is detected the gene is not FASN. In other embodiments, if expression corresponding to, or activity of protein encoded by, only one gene is detected the gene is not PLAB. For example, increased administration of the agent may be desirable to decrease the level of expression or activity of the at least one gene to lower levels than detected, i.e., to increase the effectiveness of the agent. Alternatively, decreased administration of the agent may be desirable to increase expression or activity of the at least one gene to higher levels than detected, i.e., to decrease the effectiveness of the agent.

In another aspect, a method for inhibiting undesired proliferation of a prostate cell is provided which utilizes a therapeutic agent as described above, e.g., an antisense nucleotide, a ribozyme, a double-stranded RNA, and an antagonist such as an antibody. Preferably, the prostate cell is present in a human. The undesired proliferation of the prostate cell is associated with a condition selected from the group consisting of localized prostate cancer, metastatic prostate cancer, prostatitis, benign prostatic hypertrophy and benign prostatic hyperplasia.

With respect to inhibition of proliferation of a prostate cell utilizing an antisense nucleotide, the method comprises administering to the prostate cell a therapeutically effective amount of an isolated nucleic acid molecule comprising an antisense nucleotide sequence derived from at least one gene identified in Tables 2, 3 or 4, wherein the antisense nucleotide has the ability to decrease the transcription/translation of the at least one gene. In some embodiments, if expression of only one gene is inhibited the gene is not FASN. In other embodiments, if expression of only one gene is inhibited the gene is not PLAB.

With respect to inhibition of proliferation of a prostate cell utilizing a ribozyme, such a method comprises administering to the prostate cell a therapeutically effective amount of a nucleotide sequence encoding the ribozyme, which has the ability to decrease the transcription/translation of at least one gene identified in Tables 2, 3 or 4. In some embodiments, if expression of only one gene is inhibited the gene is not FASN. In other embodiments, if expression of only one gene is inhibited the gene is not PLAB.

With respect to inhibition of proliferation of a prostate cell utilizing a double-stranded RNA, the method comprises administering to the prostate cell a therapeutically effective amount of a double-stranded RNA corresponding to at least one gene identified in Tables 2, 3 or 4, wherein the double-stranded RNA has the ability to decrease the transcription/translation of the at least one gene. In some embodiments, if expression of only one gene is inhibited the gene is not FASN. In other embodiments, if expression of only one gene is inhibited the gene is not PLAB.

With respect to inhibition of proliferation of a prostate cell utilizing an antagonist, the method comprises administering to the prostate cell a therapeutically effective amount of an antagonist that inhibits a protein encoded by at least one gene identified in Tables 2, 3 or 4. In some embodiments, if expression of only one gene is inhibited the gene is not FASN. In other embodiments, if expression of only one gene is inhibited the gene is not PLAB.

In the context of inhibiting undesired proliferation of a prostate cell, a “therapeutically effective amount” of an isolated nucleic acid molecule comprising an antisense nucleotide, a nucleotide sequence encoding a ribozyme, a double-stranded RNA, or antagonist, refers to a sufficient amount of one of these therapeutic agents to inhibit proliferation of a prostate cell (e.g., to inhibit or stabilize cellular growth of the prostate cell) and can be determined as described above.

In another aspect, a viral vector is provided which comprises a promoter and/or an enhancer or other regulatory element of a gene selected from the group consisting of at least one of the genes identified in Tables 2, 3 or 4 operably linked to the coding region of a gene that is essential for replication of the vector, wherein the vector is adapted to replicate upon transfection into a diseased prostate cell. In some embodiments, the promoter and/or an enhancer or other regulatory element of the gene is not of a FASN gene. In other embodiments, the promoter and/or enhancer or other regulatory element of the gene is not of a PLAB gene. The promoter sequences can be discerned by searching the publicly available databases for BAC clones that cover the entire gene; thereafter, the cDNA for the gene can be compared to the genomic sequence. This will generally reveal the intron-exon boundaries and the start site of the gene. Once these are established, the promoter sequences can be inferred. For FASN the promoter is well known and described in the literature, as in Wolf, Nutr. Rev., Vol. 54, No. 4, Part 1, pp.122-123 (1996); Schweizer et al., Biochem. Soc. Trans., Vol. 25, No. 4, pp. 1220-1224 (1997); and Sul et al., Prog. Nucleic Acid Res. Mol. Biol., Vol. 60, pp. 317-345 (1998). Such vectors are able to selectively replicate in a prostate cell having a disorder, but not in a non-diseased prostate cell. The replication is conditional upon the presence in a diseased prostate cell, and not in a non-diseased prostate cell, of positive transcription factors that activate the promoter of the disclosed genes. It can also occur by the absence of transcription inhibiting factors that normally occur in a non-diseased prostate cell and prevent transcription as a result of the promoter. Accordingly, when transcription occurs, it proceeds into the gene essential for replication, such that in the diseased prostate cell, but not in non-diseased prostate cell, replication of the vector and its attendant functions occur. With this vector, a diseased prostate cell, e.g., a prostate cancer, can be selectively treated, with minimal systemic toxicity.

In one embodiment, the viral vector is an adenoviral vector, which includes a coding region of a gene essential for replication of the vector, wherein the coding region is selected from the group consisting of E1a, E1b, E2 and E4 coding regions. The term “gene essential for replication” refers to a nucleic acid sequence whose transcription is required for the vector to replicate in the target cell. Preferably, the gene essential for replication is selected from the group consisting of the E1A and E1b coding sequences. Particularly preferred is the adenoviral E1A gene as the gene essential for replication. Methods for making such vectors are well know to the person of ordinary skill in the art as described, e.g., in Sambrook et al., in Molecular Cloning: A Laboratory Manual, Cold Spring Harbor, N.Y., 1989. The present invention provides novel viral vectors based on the oncolytic adenoviral vector strategy as described in U.S. Pat. No. 5,998,205, issued Dec. 7, 1999 to Hallenbeck et al. and in U.S. provisional application ______ filed Jan. 14, 2002, entitled “Novel Oncolytic Adenoviral Vectors” (Docket No. 4-31704P3/PROV/GTI), the disclosures of which are hereby incorporated by reference in their entirety. In particular, oncolytic adenoviral vectors are disclosed in which expression of at least one adenoviral gene, which is essential for replication, is controlled by a tissue-specific promoter which is selectively transactivated in cancer cells. In one embodiment a tissue-specific promoter controls the expression of E1a. In a particularly preferred embodiment both the E1a and E4 genes are controlled by tumor-specific promoters. Methods for preparing tissue-specific replication vectors and their use in the treatment of prostate cancer cells and other types of abnormal cells which are harmful or otherwise unwanted in vivo in a subject are described in detail, e.g., in U.S. Pat. No. 5,998,205. U.S. Pat. No. 5,698,443 describes adenoviral vectors, in which expression of a gene essential for replication is controlled by the PSA promoter/enhancer. Unlike the vectors of the present invention, however, the viral vectors described in this patent replicate in normal as well as diseased prostate cells, because PSA promoter/enhancer is active in normal prostate cells as well as in diseased prostate cells.

In a further embodiment, the invention provides nucleic acid constructs in which a heterologous gene product is expressed under the control of a promoter and/or an enhancer or other regulatory element of a gene selected from the group consisting of at least one of the genes identified in Tables 2, 3 or 4. In some embodiments, the promoter and/or enhancer or other regulatory element of the gene is not of a FASN gene. In other embodiments, the promoter and/or enhancer or other regulatory element of the gene is not of a PLAB gene. Such heterologous gene products are expressed when the construct is present in diseased prostate cells, but not in normal, non-diseased prostate cells. The heterologous gene product provides, in some embodiments, for the inhibition, prevention, or destruction of the growth of the diseased prostate cell, e.g., a prostate cancer. The gene product can be RNA, e.g., antisense RNA or ribozyme, or proteins such as a cytokine, e.g., interleukin, interferon, or toxins such as diphtheria toxin, pseudomonas toxin, etc. The heterologous gene product can also be a negative selective marker such as cytosine deaminase. Such negative selective markers can interact with other agents to prevent, inhibit or destroy the growth of the diseased prostate cells. U.S. Pat. No. 6,057,299, for example, describes the construction and use of nucleic acid constructs in which heterologous genes are placed under the control of a PSA enhancer. The nucleic acid constructs can be introduced into target cells by methods known to those of skill in the art. For example, one can incorporate the constructs into an appropriate vector such as those described above.

The vector of the present invention can be transfected into a helper cell line for viral replication and to generate infectious viral particles. Alternatively, transfection of the vector or other nucleic acid into a prostate cell can take place by electroporation, calcium phosphate precipitation, microinjection, or through liposomes, including proteoliposomes.

The following examples are included to demonstrate preferred embodiments of the invention.

### EXAMPLES

### Methods

Cell culture

PC3, LNCaP, and DU145 cells (American Type Culture Collection, Manassas, Va.) are grown in DME (Gibco, Rockville, Md.) supplemented with 10% fetal calf serum (FCS) and 100 μg/mL streptomycin (Gibco) (D10) at 37° C. in a humidified atmosphere of 5% CO₂ in air. LNCaP cells are also grown in androgen-depleted D10 in which dialyzed charcoal-stripped FCS is substituted for FCS. Normal prostate epithelial cells (PrEC) and an HPV E6-transfected derivative (hPr1) are grown in serum-free keratinocyte growth medium (Gibco). Fibroblastic cell strains CAF1598, CAF1303, CAF1852, and CAF2585 are expanded from fragments of prostates removed for adenocarcinoma. Frozen sections are stained with hematoxylin and eosin (H&E) at the time of surgery and determined to contain malignant epithelium; adjacent fragments are minced and plated in D1O. BPHF1598 fibroblastic cells are propagated from fragments of prostate tissue that contain only benign hypertrophic glands. Normal prostate stromal cells (PrSC) A and B are purchased from Clonetics (San Diego, Calif.) and cultured according to the manufacturer's recommendations. Adherent cells are expanded and harvested at 60-70% confluence after 4-8 passages. LNCaP, grown in the absence of androgens and two cultures of HUVEC, are each sampled twice.

Tissue Procurement, cRNA Synthesis, and Hybridization to Oligonucleotide Arrays

The use of human tissue samples at the University of Virginia is approved by the UVA Human Investigation Committee. Fresh samples of adenocarcinoma are obtained from men with elevated PSA levels. H&E-stained sections from prostatectomy specimens or (in one case) from a lymph node metastasis are examined to assess the relative amounts of tumor, benign epithelium, stroma, and lymphocytes. Tissues containing cancer are trimmed to enrich for neoplastic cells. All samples are stored at −80° C. prior to processing for microarray analysis. The set of prostate tissues include 23 primary cancer tissues, one lymph node metastasis and 9 non-neoplastic tissues. Eight of the cancers are paired with normal tissue obtained from the same patient. One cancer tissue (case 13) is divided and processed as two independent samples. In each case, several mg of tissue are sharply dissected and homogenized with a rotary homogenizer (Omni International, Warrenton, Va.) in RNeasy lysis buffer (QIAGEN, Valencia, Calif.). RNA is prepared from tissues and cells using the RNeasy Mini Kit (QIAGEN). Messenger RNAs from T-lymphoblastic MOLT4 and myeloleukemic HL60 cells are purchased from Clontech (Palo Alto, Calif.), and RNA from endothelial cells, and from three individual isolates of activated B-cells, which are gifts from A. Kawamura and M. Cooke, respectively. Labeled complementary RNA (cRNA) is prepared and hybridized to “U95A” oligonucleotide arrays (Affymetrix, Santa Clara, Calif.) as previously described in Lockhart et al., Nat. Biotechnol., Vol. 14, pp. 1675-1680 (1996); and Wodicka et al., Nat. Biotechnol., Vol. 15, pp. 1359-1367 (1997).

Data Analysis

Scanned image files are visually inspected for artifacts and analyzed with GENECHIP 3.1 (Affymetrix). Each image is then scaled to an average hybridization intensity of 200, which corresponds to ˜3-5 transcripts per cell as described in Wodicka et al., supra. The hybridization intensity for each gene is ranked according to inter-sample variability (standard deviation), and 3,530 genes with the most variable expression across all of the samples are median-centered and normalized with respect to other genes in the sample and corresponding genes in other samples. Genes and samples are subjected to hierarchical clustering essentially as described in Eisen et al., Proc. Natl. Acad. Sci. USA, Vol. 95, pp. 14863-14868 (1998). Differential expression of genes in benign and malignant prostate tissues are estimated by comparing the hybridization intensity of each gene, using three different estimates for population differences (difference of means, quotient of means, and unpaired t test). The genes are ranked according to each metric and the sum of the ranks is used as a semiquantitative estimate of the differential abundance of each transcript.

RT-PCR Analysis of Hepsin and PLAB Expression

cDNA is prepared using 1 μg total RNA isolated from prostate tissues. Primers used to amplify specific gene products are: hepsin sense, 5′-CGGGACCCCMCAGCGAGGAGMC-3′; hepsin antisense, 5′-TCGGGGTAGCCAGCACAGMCATC-3′; PLAB sense, 5′-CGCGCMCGGGGACGACT-3′; and PLAB antisense, 5′-TGAGCACCATGGGATTGTAGC-3′. PCR conditions for hepsin and PLAB comprise 95° C. for 10 minutes, 30 cycles of 95° C. for 30 seconds, 55° C. for 30 seconds (annealing), and 72° C. for 30 seconds, and a final elongation step of 72° C. for 7 minutes. All PCR reactions use a volume of 20 μL, with 1 U AmpliTaq Gold (Perkin-Elmer, Foster City, Calif.). Amplification products (10 μL) are separated by 2% agarose gel electrophoresis.

Immunohistochemistry

The avidin-biotin immunoperoxidase method is performed on deparaffinized zinc formalin-fixed, paraffin-embedded sections. Slides placed in citrate buffer are heated with a microwave for 20 minutes prior to the application of the anti-fatty acid synthase antibody (a gift from E. Pizer) for 1 hour at room temperature.

### Example 1

### Gene Expression Profiles in Prostate Tissue Samples and Cell Lines

To test the hypothesis that gene expression profiles could be used to classify prostate tissue samples, the expression levels of genes in tissues and cells is monitored by hybridization of RNA samples to oligonucleotide microarrays representing approximately 8,920 different genes. In total, 55 RNA samples derived from 25 prostate cancer tissues (24 unique samples), nine nonmalignant prostate tissues, and 21 cell line samples of various origin (18 unique lines) (see Materials and Methods) are hybridized. The complete dataset is available on the web site (http://www.gnf.org/cancer/prostate).

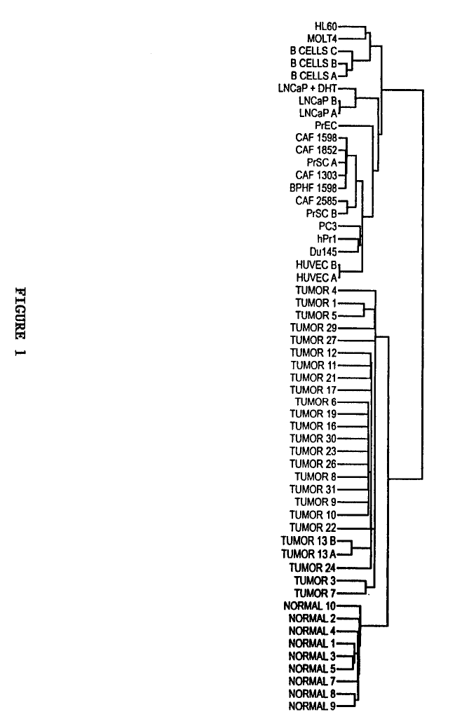

To reveal distinctions between individual tissue samples and cells, a subset of 3,530 genes that have varied most across the samples is selected and grouped both the genes and the samples according to their overall similarities in levels of expression as described, e.g., in Eisen et ai., Proc. Natl. Acad. Sci. USA, Vol. 95, pp. 14863-14868 (1998). These samples are separated on a dendrogram in which their overall similarity is proportional to the length of the vertical branches between them (FIG. 1). As expected, replicate samples of tumor 13, LNCaP cells, and umbilical vein endothelial cells (HUVEC) are highly correlated, serving as an internal validation of the technique. This analysis reveals a major division between cells grown in vitro and the human tissue specimens, with highly divergent gene expression patterns. Within the “tissue” branch of the dendrogram, samples are further subdivided into two distinct groups. The first is composed entirely of the 24 prostate tumors and the second includes all nine nonmalignant samples. Thus, analysis of gene expression faithfully reproduces a distinction based on the presence or absence of malignant epithelium.

Within the “cell line” branch of the dendrogram, a high degree of relatedness is identified among cells with different tissue origins. For example, profiles of activated B-cells from three independent donors are highly correlated with one another and with cells derived from two hematologic cancers (MOLT4 and HL60) (FIG. 1). Similarly, six stromal fibroblast lines derived from normal or malignant prostates of different individuals (designated as BPHF, PrSC, or CAF) are highly related. Similarity is also seen among LNCaP cells grown in the presence or absence of dihydrotestosterone (DHT). PC3 and Du145 cells, derived from metastatic prostate lesions as androgen-independent lines, are grouped together with immortal, androgen-independent basal prostate epithelial cells transfected with the HPV E6 gene (hPr1). Normal basal prostate epithelial cells (PrEC) cells, by contrast, are isolated on a branch of the dendrogram between the LNCaP cells and DU145, hPr1 and PC3 cells. The separation of cells from tissues is due in large part to the differential expression of a large cluster of genes associated with cell cycle progression including cyclin B, cdc2, and cks1, reflecting the rapid growth rates of cells grown in vitro. This is corroborated by the low or absent expression of these genes in androgen-deprived LNCaP cells (duplicate “LNCaP -DHT,” FIG. 1) which arrest in the G1 phase of the cell cycle under these conditions as described, e.g., in Esquenet et al., Prostate, Vol. 28, pp. 182-194 (1996).

### Example 2

### Expression of Genes From Different Cell Types

Prostate cancer samples contain varying fractions of non-malignant epithelial cells, fibromuscular stroma, endothelium, and infiltrating immune cells. Representatives of these cell types are profiled to help resolve cell type-specific expression patterns. From among the 3,530 genes analyzed, four major clusters are identified for which expression levels were similar in some of the cells and tissues. One cluster contains genes whose expression is high in stromal cells grown in vitro and in normal prostate samples, which microscopically contain a significant amount of stroma (range, 10-50%). This cluster includes genes for the α1 and α2 subunits of collagen VI and the fibroblast growth factor receptor, Type 1 (FGF-R1), which are expressed predominantly in fibroblasts. The concordance of stromal genes by this approach is not complete, as other groups of stromal genes within the primary tissues are identified that are absent or expressed at low levels in some of the stromal cells. These discrepancies are attributed to the selection of prostate tissue fibroblasts for growth in culture, in which differentiated fibromuscular cells are unlikely to have been favored for rapid proliferation. A second concordant “tissue-cell” cluster contains a large number of immunoglobulin (Ig) genes, which are expressed by activated B-cells and some cells in several of the benign and malignant tissues. Paired normal and malignant samples from cases 9 and 10 express very high levels of these genes, which are consistent with a histological finding of prostatitis in these two patients. The third cluster contains genes characteristically expressed by basal epithelial cells, such as keratins 5 and 17 (Peehl et al., Cell Tissue Res., Vol. 277, pp. 11-18 (1994); and Troyanovsky et al., Eur. J. Cell Biol., Vol. 59, pp. 127-137 (1992), respectively). Here, concordant gene expression is found for a set of these genes in PrEC and hPr1 cells (known to be basal epithelial cells as described in Choo et al., Prostate, Vol. 40, pp. 150-158 (1999)), and in normal and tumor tissues to variable extents. The fourth group of genes are highly expressed in LNCaP cells and in most of the tumor samples and include multiple genes whose products are involved in intermediary metabolism, such as ATP synthase and cytochrome C oxidase. Expression of genes within this cluster likely reflects accelerated growth of the malignant cells.

In contrast to the concordant “tissue-cell” clusters described above, other groups of genes specifically expressed by T-cells and endothelial cells are identified which are not highly expressed in any of the tissue samples. There is also minimal concordance between gene expression levels in prostate tissues and the three epithelial prostate cancer cell lines that are profiled (LNCaP, PC3 and DU145). A group of approximately 400 genes is highly and specifically expressed in malignant samples and includes keratins 8 and 18 which signify malignant luminal epithelium in cancer tissue. Over-expression of these genes contributes substantially to the molecular distinction between benign and malignant tissues. This group includes hK2, the epidermal growth factor receptor (ERBB3), prostate-specific membrane antigen (PSMA), the androgen receptor (AR), and three isoforms of PSA.

### Example 3

### Differences in Gene Expression Among Prostate Tumors

The apparent molecular similarity among prostate results from the initial analysis of 3,530 genes whose expression varied most across normal tissues, malignant tissues, and cell lines. To better estimate the extent of the similarity among prostate cancers, 788 of these genes are selected whose expression is inferred to be specific to the malignant cells (the list of genes and the criteria used to select them are available at our web site). Expression levels of this group of tumor-specific genes are used to select for a smaller group (n=277) that vary most significantly across the tumors in an attempt to magnify any potential taxonomy. Clustering of the data shows a dichotomy among the tumors, which is largely attributable to differential expression of a group of ribosomal genes. This division roughly corresponds to the tumors' degree of differentiation, with significantly higher Gleason scores (P<0.01) in the tumor samples with lower ribosomal gene expression.

The single lymph node metastasis (case 17) is not readily distinguished from the primary tumors by reclustering, but expresses high levels of multiple interferon-responsive genes which are not highly expressed in any other sample and includes the interferon-inducible proteins p27 and p78. These genes are likely expressed by malignant cells, as the specimen contains at least 90% tumor cells by histology and covariant expression of these genes is not found in any of the profiled cell lines.

### Example 4

### Identification of Potential Diagnostic Markers and Therapeutic Targets

The molecular homogeneity of prostate cancers when compared to normal prostate tissues suggests that genes with potential clinical utility can be identified that are over-expressed in the majority of tumors. Genes are first ranked, based on the arithmetic difference in expression level between benign and malignant samples (the “difference” metric, FIG. 2a). Multiple probe sets representing isoforms of PSA and hK2 are the most highly ranked by this method. The approach highlights two key problems in the use of genes or gene products such as PSA as diagnostic markers. First, the ratio of PSA transcript levels is modest when comparing normal and malignant samples (˜1.6-fold). Second, the range of expression of PSA within the groups of tissues is large, and levels in normal and malignant tissues often coincides. Hence, a metric is devised to select for genes whose expression is low in normal tissues, high in malignant tissues, and whose ranges of expression in normal, and malignant samples are well separated from one another as described in Su and Hampton, U.S. Provisional Application No. 60/297,277, entitled “Molecular Signatures of Commonly Fatal Carcinomas,” which was filed on Jun. 10, 2001. (See, also, Welsh et al., Cancer Res., Vol. 61, pp. 5974-5978 (2001)) This metric returns genes with idealized “diagnostic” profiles, including known tumor antigens, such carcinoma-associated antigen GA733-2 (TACSTD1), intestinal trefoil factor 3 (TFF3), and fatty acid synthase (FASN). It also identifies strong differential expression of PLAB (prostate differentiation factor) which encodes a secreted cytokine as described in Bootcov et al., Proc. Natl. Acad. Sci. USA, Vol. 94, pp. 11514-11519 (1997); and hepsin, which encodes a membrane-bound extracellular serine protease involved in cell growth control as described in Torres-Rosado et al., supra. (FIG. 2b). Arbitrary expression levels of FASN, PLAB and hepsin transcripts are imposed to demonstrate separation of normal from malignant tissues as shown in Table 1 below.

TABLE 1
Selected Transcript Levels in Normal and Malignant Prostate Tissues

| | | | | FASN | PLAB | Hepsin |
|---|---|---|---|---|---|---|
| Prostate histology | Grade | Cases | Stage | >1000 | >1000 | >500 |
| Non-cancerous, no adjacent | — | 1 | — | 0 (0%) | 0 (0%) | 0 (0%) |
| tumor | | | | | | |
| Non-cancerous, near tumor | — | 8 | — | 0 (0%) | 2 (25%) | 0 (0%) |
| Primary adenocarcinoma | 5 | 1 | 2a | 1 (100%) | 1 (100%) | 1 (100%) |
| | 6 | 7 | 2b-3b | 7 (100%) | 6 (86%) | 7 (100%) |
| | 7 | 9 | 2a-3b | 9 (100%) | 8 (89%) | 9 (100%) |
| | 8 | 4 | 2a-3 | 4 (100%) | 3 (75%) | 4 (100%) |
| | 9 | 2 | 2b | 2 (100%) | 2 (100%) | 2 (100%) |
| Metastatis to lymph node | 8 | 1 | 4 | 0 (0%) | 1 (100%) | 1 (100%) |
| Adenocarcinoma, total | — | 24 | 2a-4 | 23 (96%) | 21 (88%) | 24 (100%) |
| cases | | | | | | |

Levels are given as hybridization intensities.
Stage is given as ranges for the cancers.

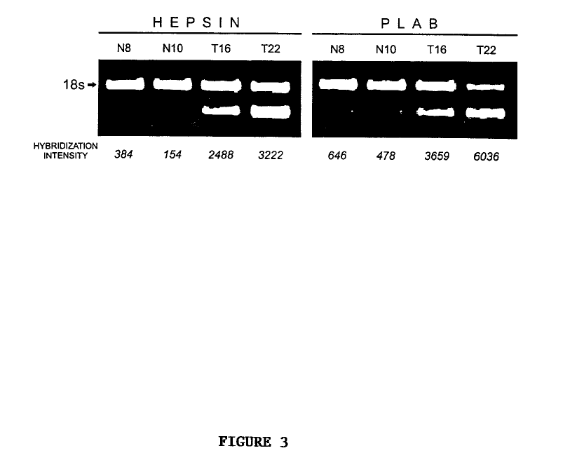

The 20 highest-ranked genes (those in FIG. 3b) are tabulated below (Table 2).

TABLE 2
List of Top 20 Genes Identified According to Metric

| Accession no. | Gene name | AVG_NL | AVG_T |
|---|---|---|---|
| X07732 | HEPSIN | 259.2 | 1728.6 |
| AJ130733 | MeAcCoA-RACEMASE | 150.6 | 1624.6 |
| AB000584 | TGF-βSF | 690.8 | 4657.5 |
| AF061258 | LIM | 510.2 | 2030.8 |
| AL049969 | UNKNOWN (AL049969) | 1228.6 | 3874.0 |
| AF065388 | NET-1 | 1073.0 | 3464.4 |
| U80456 | SIM2 | 26.4 | 410.9 |
| M26326 | KRT18 | 875.1 | 2629.8 |
| M77836 | PYRROLINE | 35.0 | 367.7 |
| | REDUCTASE | | |
| M93036 | GA733-2 | 307.0 | 1030.6 |
| AF052107 | UNKNOWN (AF052107) | 121.6 | 598.1 |
| U29344 | BrCa FA SYNTHASE | 661.8 | 2005.1 |
| AF071202 | MOAT-B | 154.7 | 724.8 |
| AJ223352 | HISTONE H2B | 413.8 | 1320.7 |
| AB018330 | UNKNOWN (AB018330) | 440.8 | 1434.7 |
| X70326 | MacMARCKS | 1282.6 | 3338.4 |
| U83660 | MRP4 | 84.8 | 540.8 |
| X83425 | LUTHERAN | 499.7 | 1427.8 |
| L08044 | TREFOIL FACTOR | 371.6 | 3447.7 |
| U07919 | ADH6 | 1370.7 | 3253.5 |

(Notes: Accession number can be used to identify the unique identity of each gene at NCBI - UniGene at http:f/www.ncbi.nlm.nih.gov/UniGene/; AVG_NL and AVG_TUMOR are the average of the average difference hybridization intensities in normal and tumor tissues, respectively.)

The differential expression of genes that are ranked highly by this method are validated by RT-PCR amplification of PLAB and hepsin transcripts in selected RNAs from normal and cancer tissues, confirming their over-expression in prostate cancers (FIG. 3). The public “Gene-to-Tag” database (http://www.ncbi.nlm.nih.gov/SAGE/) is also queried for differential expression of 20 highly-ranked genes (those in FIG. 2b and Table 2) in normal and prostate cancer samples. Reliable estimates of the expression levels in a single microdissected pair of normal and malignant prostate samples are available for 12 of the 20 genes. Of these 12, ten show greater than 5-fold over-expression in cancer tissue. Differential expression is also confirmed using a monoclonal antibody against FASN (Pizer et al., Prostate, Vol. 47, pp. 102-110 (2001)) in immunohistochemical staining, and found strong and specific immunopositivity in malignant epithelium in all 10 cases of prostate cancer that we examined. In addition to the 20 genes listed in Table 2, two isoforms of PSA are highly ranked with respect to differential expression in prostate tumors, as shown below in Table 3 (see also FIG. 2a).

TABLE 3
List of Two Isoforms of PSA Identified According to “Difference” Metric

| Accession no. | Gene name | AVG_NL | AVG_T |
|---|---|---|---|
| HT2351 | Prostate specific antigen, | 3466 | 8607 |
| | alternative splice form 2 | | |
| HT2352 | Prostate specific antigen, | 6610 | 12699 |
| | alternative splice form 3 | | |

(Notes: Accession number can be used to identify the unique identity of each gene at TIGR at http://www.tigr.org; AVG_NL and AVG_TUMOR are the average of the average difference hybridization intensities in normal and tumor tissues, respectively.)

Table 4 below includes the 20 genes identified in Tables 2 and 3 and other genes highly ranked by the “difference” metric.

TABLE 4
List of Top 50 Genes Identified According to Metric

| | | Ave. expression | Ave. expression |
|---|---|---|---|
| Accession no. | Gene name | normal | tumor |
| X07732 | hepsin | 259 | 1832 |
| AJ130733 | 2-methylacyl-CoA racemase | 151 | 1735 |
| AB000584 | MIC-1 | 691 | 4924 |
| AF061258 | LIM protein | 510 | 2146 |
| AL049969 | Unknown | 1229 | 4091 |
| AF065388 | NET-1 | 1073 | 3631 |
| M77836 | pyrroline 5-carboxylate reductase | 35 | 394 |
| M26326 | Keratin 18 | 875 | 2745 |
| U80456 | SIM2 | 26 | 435 |
| M93036 | GA733-2 | 307 | 1083 |
| AF052107 | Unknown | 122 | 636 |
| U29344 | Fatty acid synthase | 662 | 2080 |
| X70326 | MacMarcks | 1283 | 3492 |
| AF071202 | MRP4 | 155 | 766 |
| AJ223352 | histone H2B | 414 | 1376 |
| AB018330 | K1AA0787 | 441 | 1509 |
| U83660 | MRP4 | 85 | 578 |
| X83425 | Lutheran | 500 | 1479 |
| M30894 | T-cell receptor Ti rearranged | 1495 | 5025 |
| | gamma-chain | | |
| U07919 | aldehyde dehydrogenase 6 | 1371 | 3387 |
| D82345 | NB thymosin beta | 230 | 957 |
| AL079298 | Unknown | 169 | 811 |
| HT2351 | Prostate Specific, Alt. Splice Form 2 | 3466 | 8594 |
| W29087 | Unknown | 247 | 741 |
| HT2352 | Antigen, Prostate Specific, Alt. Splice | 472 | 1361 |
| | Form 3 | | |
| AL039458 | Unknown | 459 | 1187 |
| J02783 | thyroid hormone binding protein (p55) | 2359 | 5171 |
| Y00486 | adenine phosphoribosyltransferase | 183 | 577 |
| L08044 | intestinal trefoil factor | 372 | 3623 |
| AJ002308 | synaptogyrin 2 | 2095 | 4520 |
| U52522 | arfaptin 2 | 289 | 802 |
| X73066 | Unknonw | 332 | 869 |
| AC005053 | Unknown | 922 | 2350 |
| HT2351 | Antigen, Prostate Specific, Alt. Splice | 3088 | 7319 |
| | Form 2 | | |
| M22806 | thyroid hormone binding protein (p55) | 3648 | 7540 |
| A1885852 | Unknown | 969 | 2926 |
| X87176 | 17-beta-hydroxysteroid | 194 | 657 |
| | dehydrogenase | | |
| M64788 | GTPase activating protein (rap1GAP) | 90 | 390 |
| AF039918 | CD39L4 | 55 | 394 |
| US1903 | RasGAP-related protein (IQGAP2) | 92 | 344 |
| Z80776 | Histone H2A/g | 349 | 971 |
| AC003034 | Unknown | 91 | 473 |
| U21931 | fructose-1,6-biphosphatase | 333 | 947 |
| A1198311 | Unknown | 963 | 5033 |
| AL109672 | Unknown | 468 | 1122 |
| A1039144 | Unknown | 12 | 292 |
| AL049977 | Unknown | 80 | 462 |
| S82986 | HOXC6 | 20 | 176 |
| U21090 | DNA polymerase delta small subunit | 205 | 535 |
| D13748 | eukaryotic initiation factor 4A1 | 1140 | 2271 |

(Notes; Accession number can be used to identify the unique identity of each gene at NCBI - UniGene at http://www.ncbi.nlm.nlh.gov/UniGene/ and at TIGR at http://www.tigr.org or http://www.ncbi.nlm.nih.gov/Entrez; AVG_NL and AVG_TUMOR are the average of the average difference hybridization intensities in normal and tumor tissues, respectively.)

It will be understood that various modifications may be made to the embodiments and/or examples disclosed herein. Thus, the above description should not be construed as limiting, but merely as exemplification of preferred embodiments. Those skilled in the art will envision other modifications within the scope and spirit of the claims appended hereto.

We claim:

1. A method for screening a subject for a prostate disorder or at risk of developing a prostate disorder, the method comprising:
a) detecting a level of expression of at least one gene identified in Tables 2, 3 or 4 in a sample of prostate tissue obtained from the subject to provide a first value, with the proviso that if expression of only one gene is detected that the gene is not FASN; and
b) comparing the first value with a level of expression of the at least one gene identified in Tables 2, 3 or 4 in a sample of prostate tissue obtained from a disease-free subject, wherein a greater expression level in the subject sample compared to the sample from the disease-free subject is indicative of the subject having a prostate disorder or at risk of developing a prostate disorder.

2. The method of claim 1, wherein the level of expression of at least two genes identified in Tables 2, 3 or 4 are detected.

3. The method of claim 1, wherein the at least one gene identified in Tables 2, 3 or 4 is selected from the group consisting of hepsin, prostate differentiation factor, alpha-methylacyl-CoA racemase, fatty acid synthase, prostate specific antigen, alternative splice form 2 and prostate specific antigen, alternative splice form 3.

4. The method of claim 1, wherein the prostate disorder is selected from the group consisting of localized prostate cancer, metastatic prostate cancer, prostatitis, benign prostatic hypertrophy and benign prostatic hyperplasia.

5. The method of claim 1, wherein the level of expression of the gene is determined by detecting the level of expression of a mRNA corresponding to the gene.

6. The method of claim 5, wherein the level of expression of mRNA is detected by techniques selected from the group consisting of Northern blot analysis, reverse transcription PCR and real time quantitative PCR.

7. The method of claim 1, wherein the level of expression of the gene is determined by detecting the level of expression of a protein encoded by the gene.

8. The method of claim 7, wherein the level of expression of the protein is detected through western blotting by utilizing a labeled probe specific for the protein.

9. The method of claim 8, wherein the probe is an antibody.

10. The method of claim 9, wherein the antibody is a monoclonal antibody.

11. A method for monitoring the progression of a prostate disorder in a subject having, or at risk of having, a prostate disorder comprising measuring a level of expression of at least one gene identified in Tables 2, 3 or 4 over time in a prostate tissue sample obtained from the subject with the proviso that if expression of only one gene is detected that the gene is not FASN, wherein an increase in the level of expression of the at least one gene over time is indicative of the progression of the prostate disorder in the subject.

12. The method of claim 11, wherein the level of expression of at least two genes identified in Tables 2, 3 or 4 is measured.

13. The method of claim 11, wherein the at least one gene identified in Tables 2, 3 or 4 is selected from the group consisting of hepsin, prostate differentiation factor, alpha-methylacyl-CoA racemase and fatty acid synthase, prostate specific antigen, alternative splice form 2 and prostate specific antigen, alternative splice form 3.

14. The method of claim 11, wherein the prostate disorder is selected from the group consisting of localized prostate cancer, metastatic prostate cancer, prostatitis, benign prostatic hypertrophy and benign prostatic hyperplasia.

15. The method of claim 11, wherein the level of expression of the gene is determined by detecting the level of expression of a mRNA corresponding to the gene.

16. The method of claim 15, wherein the level of expression of mRNA is detected by techniques selected from the group consisting of Northern blot analysis, reverse transcription PCR and real time quantitative PCR.

17. The method of claim 11, wherein the level of expression of the gene is determined by detecting the level of expression of a protein encoded by the gene.

18. The method of claim 17, wherein the level of expression of the protein encoded by the gene is detected through western blotting by utilizing a labeled probe specific for the protein.

19. The method of claim 18, wherein the labeled probe is an antibody.

20. The method of claim 19, wherein the antibody is a monoclonal antibody.

21. A method for identifying agents for use in the treatment of a prostate disorder comprising:
a) contacting a sample of diseased prostate cells with a candidate agent;
b) detecting a level of expression of at least one gene in the diseased prostate cells, wherein the at least one gene is identified in Tables 2, 3 or 4, with the proviso that if expression of only one gene is detected that the gene is not FASN; and
c) comparing the level of expression of the at least one gene in the sample in the presence of the candidate agent with a level of expression of the at least one gene in cells that are not contacted with the candidate agent, wherein a decreased level of expression of the at least one gene in the sample in the presence of the candidate agent relative to the level of expression of the at least one gene in the sample in the absence of the candidate agent is indicative of an agent useful in the treatment of a prostate disorder.

22. The method of claim 21 wherein the level of expression of at least two genes in the sample is detected in step (b).

23. The method of claim 21, wherein the at least one gene identified in Tables 2, 3 or 4 is selected from the group consisting hepsin, prostate differentiation factor, alpha-methylacyl-CoA racemase, fatty acid synthase, prostate specific antigen, alternative splice form 2 and prostate specific antigen, alternative splice form 3.

24. The method of claim 21, wherein the prostate disorder is selected from the group consisting of localized prostate cancer, metastatic prostate cancer, prostatitis, benign prostatic hypertrophy and benign prostatic hyperplasia.

25. The method of claim 21, wherein the level of expression of the gene is determined by detecting the level of expression of a mRNA corresponding to the gene.

26. The method of claim 25, wherein the level of expression of mRNA is detected by techniques selected from the group consisting of Northern blot analysis, reverse transcription PCR and real time quantitative PCR.

27. The method of claim 21, wherein the agent is selected from the group consisting of antisense nucleotides, ribozymes and double stranded RNAs.

28. The method of claim 21, wherein the level of expression of the gene is determined by detecting the level of expression of a protein encoded by the gene.

29. The method of claim 28, wherein the level of expression of the protein encoded by the at least one gene is detected through western blotting by utilizing a labeled probe specific for the protein.

30. The method of claim 29, wherein the labeled probe is an antibody.

31. The method of claim 30, wherein the antibody is a monoclonal antibody.

32. A method of inhibiting undesired proliferation of a prostate cell, the method comprising administering to the cell an effective amount of an agent that can decrease the expression of at least one gene identified in Tables 2, 3 or 4, with the proviso that if expression of only one gene is inhibited that the gene is not FASN.

33. The method of claim 32, wherein the agent is selected from the group consisting of antisense nucleotides, ribozymes and double stranded RNAs.

34. The method of claim 33, wherein the agent comprises an isolated nucleic acid molecule comprising an antisense nucleotide sequence derived from at least one gene identified in Tables 2, 3 or 4.

35. The method of claim 34, wherein antisense nucleotide sequences are derived from at least two genes identified in Tables 2, 3 or 4.

36. The method of claim 32, wherein the at least one gene is selected from the group consisting of hepsin, prostate differentiation factor, alpha-methylacyl-CoA racemase, fatty acid synthase, prostate specific antigen, alternative splice form 2 and prostate specific antigen, alternative splice form 3.

37. The method of claim 32, wherein the undesired proliferation is associated with a condition selected from the group consisting of localized prostate cancer, metastatic prostate cancer, prostatitis, benign prostatic hypertrophy and benign prostatic hyperplasia.

38. The method of claim 32, wherein the agent is an antagonist that inhibits a protein encoded by at least one gene identified in Tables 2, 3 or 4.

39. The method of claim 38, wherein the at least one gene is selected from the group consisting of hepsin, FASN and MOAT-B.

40. The method of claim 38, wherein the antagonist is an antibody specific for the protein.

41. The method of claim 40, wherein the antibody is a monoclonal antibody.

42. The method of claim 40, wherein the monoclonal antibody is conjugated to a toxic reagent.

43. The method of claim 32, wherein the cell is present in a human.

44. A method for monitoring the efficacy of a treatment of a subject having a prostate disorder or at risk of developing a prostate disorder with an agent, the method comprising:
a) obtaining a pre-administration sample from the subject prior to administration of the agent;
b) detecting a level of expression of at least one gene identified in Tables 2, 3 or 4 in the pre-administration sample, with the proviso that if expression of only one gene is detected that the gene is not FASN;
c) obtaining one or more post-administration samples from the subject;
d) detecting a level of expression of the at least one gene in the post-administration sample or samples;
e) comparing the level of expression of the at least one gene in the pre-administration sample with the level of expression of the at least one gene in the post- administration sample; and
f) adjusting the administration of the agent accordingly.

45. The method of claim 44, wherein the level of expression of at least two genes identified in Tables 2, 3 or 4 is detected in step (b).

46. The method of claim 44, wherein the at least one gene is selected from the group consisting of hepsin, prostate differentiation factor, alpha-methylacyl-CoA racemase, fatty acid synthase, prostate specific antigen, alternative splice form 2 and prostate specific antigen, alternative splice form 3.

47. The method of claim 44, wherein the prostate disorder is selected from the group consisting of localized prostate cancer, metastatic prostate cancer, prostatitis, benign prostatic hypertrophy and benign prostatic hyperplasia.

48. The method of claim 44, wherein the level of expression of the gene is determined by detecting the level of expression of a mRNA corresponding to the gene.

49. The method of claim 48, wherein the level of expression of mRNA is detected by techniques selected from the group consisting of Northern blot analysis, reverse transcription PCR and real time quantitative PCR.

50. The method of claim 44, wherein the level of expression of the gene is determined by detecting the level of expression of protein encoded by the gene.

51. The method of claim 50, wherein the level of expression of protein encoded by the gene is detected through western blotting by utilizing a labeled probe specific for the protein.

52. The method of claim 51, wherein the labeled probe is an antibody.

53. The method of claim 52, wherein the antibody is a monoclonal antibody.

54. A viral vector comprising a promoter and/or enhancer of at least one gene selected from the group consisting of the genes identified in Tables 2, 3 or 4 with the proviso that the gene is not FASN, operably linked to a coding region of a gene that is essential for replication of the vector, wherein the vector is adapted to replicate upon transfection into a diseased prostate cell.

55. The vector of claim 54, wherein the viral vector is an adenoviral vector.

56. The vector of claim 54, wherein the coding region of the gene essential for replication of the vector is selected from the group consisting of E1a, E1b, E2 and E4 coding regions.

57. The vector of claim 54, further comprising a nucleotide sequence encoding a heterologous gene product.

58. A nucleic acid construct that comprises a promoter and/or enhancer of at least one gene selected from the group consisting of the genes identified in Tables 2, 3 or 4 with the proviso that the gene is not FASN, operably linked to a coding region of a heterologous gene product.

59. The nucleic acid construct of claim 58, wherein the heterologous gene product is an RNA molecule.

60. The nucleic acid construct of claim 59, wherein the RNA molecule is an antisense RNA or a ribozyme.

61. The nucleic acid construct of claim 58, wherein the heterologous gene product is a protein.

62. The nucleic acid construct of claim 61, wherein the protein is selected from the group consisting of a cytokine or a toxin.
